(19) United States
(12) Patent Application Publication
(10) Pub. No.: US 2024/0138843 A1
(43) Pub. Date: May 2, 2024

(54) IMPLANT DETACHMENT SYSTEMS WITH A MODIFIED PULL WIRE

(71) Applicant: DePuy Synthes Products, Inc., Raynham, MA (US)

(72) Inventors: Ariel SOTO DEL VALLE, Miami, FL (US); Lacey GOROCHOW, Miami, FL (US)

(73) Assignee: DePuy Synthes Products, Inc., Raynham, MA (US)

(21) Appl. No.: 18/411,290

(22) Filed: Jan. 12, 2024

Related U.S. Application Data
(63) Continuation of application No. 17/566,287, filed on Dec. 30, 2021.

(51) Int. Cl. A61B 17/12

(52) U.S. Cl. CPC A61B 17/12113; A61B 17/1215; A61B 2017/12054

(57) ABSTRACT

A detachment system for delivering an embolic coil implant to a treatment site is provided. A pull wire through a lumen of the detachment system that engages a loop wire can include a slack section. The slack section can be one or more bends, a spiral coil, or a stretchable material. The slack section is effective to inhibit premature detachment of the implantable medical device by inhibiting movement of the distal end of the pull wire when the detachment system is traversing a microcatheter.

### CROSS REFERENCE TO RELATED APPLICATIONS

The present application is a continuation application of U.S. patent application Ser. No. 17/566,287 filed Dec. 30, 2021. The entire contents of which are hereby incorporated by reference.

### FIELD OF INVENTION

This invention generally relates to intravascular medical device systems that navigate through body vessels of a human subject and, more particularly, to detachment/delivery systems for delivering and deploying an implantable medical device to a target location of a body vessel and methods of using the same.

### BACKGROUND

Aneurysms can be intravascularly treated by delivering a treatment device to the aneurysm to fill the sac of the aneurysm with embolic material and/or block the neck of the aneurysm to inhibit blood flow into the aneurysm. When filling the aneurysm sac, the embolic material can promote blood clotting to create a thrombotic mass within the aneurysm. When treating the aneurysm neck without substantially filling the aneurysm sac, blood flow into the neck of the aneurysm can be inhibited to induce venous stasis in the aneurysm and facilitate natural formation of a thrombotic mass within the aneurysm.

In some current treatments, multiple embolic coils are used to either fill the aneurysm sac or treat the entrance of the aneurysm neck. A common challenge among embolic coil treatments is that implanted coils and implanted portions of partially implanted coils can become entangled and difficult to reposition. In some instances, a physician may not be able to retract a partially implanted coil and may be forced to position the coil in a non-ideal location. Improperly positioning embolic coils at the aneurysm neck can potentially have the adverse effect of impeding the flow of blood in the adjoining blood vessel, particularly if the entrance and/or sac is overpacked. If a portion of the improperly placed coil becomes dislodged, it can enter the neighboring blood vessel and promote clot formation, which can ultimately lead to an obstruction that is tethered to the aneurysm and therefor extremely difficult to treat. Conversely, if the entrance and/or sac is insufficiently packed, blood flow can persist into the aneurysm.

In some current treatments, an embolic coil is attached to a tubular delivery device and delivered via a delivery catheter to an aneurysm. During delivery, the embolic coil can be engaged to the delivery member's implant detachment/deployment system (referred to herein equivalently as an “detachment system” or “deployment system”). When the embolic coil is in position, the deployment system can release the coil, the coil can be left implanted, and the delivery member can be retracted. Some treatments utilize a mechanical detachment/deployment system that can be actuated by a physician to release the implant by pulling one or more wires or other elongated members referred to generically herein as a “pull wire.” Some of the challenges that have been associated with delivering and deploying embolic coils with delivery members having mechanical detachment systems include premature release of a coil due to and premature movement of the pull wire proximally, thereby releasing the coil before the system is at the treatment site. This is exacerbated because of the system moves though tortuous vasculature to the treatment site.

There is therefore a need for improved methods, devices, and systems to facilitate implantation of embolic coils and other implants facing similar challenges.

### SUMMARY

It is an object of the present invention to provide systems, devices, and methods to meet the above-stated needs. In some examples presented herein, premature proximal movement or translation of a pull wire can be decreased by providing a pull wire having a slack section that allows a proximal portion of the pull wire to move independent of the distal end of the pull wire, thereby enabling a certain tolerance to proximal movement to prevent premature retraction of the pull wire through the loop wire.

A detachment system for delivering an implantable medical device to a target location of a body vessel can include a tubular body comprising a lumen extending therethrough and a compressed distal tube. The detachment system can include a loop wire comprising a first end affixed to the tubular body and comprising a loop opening positioned proximate a distal end of the compressed distal tube. The detachment system can include a pull wire extending through the lumen and through the loop opening. The pull wire can include a slack section positioned within the lumen enabling a proximal end of the pull wire to translate proximally and distally independently from a distal end of the pull wire when the detachment system is traversing a microcatheter.

The slack section can have a first length when the pull wire is in a relaxed condition and a second length when the pull wire is in a tensed condition. The distal end of the pull wire can translate proximally when the slack section exceeds the second length.

The slack section is can inhibit premature detachment of the implantable medical device by inhibiting movement of the distal end of the pull wire when the detachment system is traversing a microcatheter.

The slack section can be a plurality of bends in the pull wire positioned proximal to a location wherein the loop wire contacts the pull wire.

The slack section can be a spiral coil formed into the pull wire proximal to a location wherein the loop wire contacts the pull wire.

The slack section can be a section of stretchable material positioned along a length of the pull wire proximal to a location wherein the loop wire contacts the pull wire.

The pull wire can be defined by a proximal rigid section and a distal rigid section, and the slack section—including the stretchable material—can be positioned between the proximal rigid section and the distal rigid section, wherein the proximal rigid section and the distal rigid section as a lower degree of elasticity than the section of stretchable material.

The section of stretchable material can be a polymeric suture.

The detachment system can include a key affixed to the implantable medical device proximate a proximal end of the implantable medical device. The key can include a distal opening therethrough, wherein a stretch resistant fiber passes through the distal opening. The key can include a proximal opening therethrough. The key can include a bridge separating the distal opening and the proximal opening. The stretch resistant fiber can be engaged to the key, extended through an implant lumen of the implantable medical device, and affixed to the implantable medical device proximate a distal end of the implantable medical device. The slack section can be positioned proximal to the key.

A detachment system for delivering an implantable medical device to a target location of a body vessel can include a pull wire extending through a tubular body of the detachment system. The detachment system can include a loop wire looped over the pull wire at a distal end of the loop wire. The pull wire can include a slack section positioned proximal to a loop opening in the loop wire. The slack section can inhibit premature detachment of the implantable medical device by inhibiting movement of a distal end of the pull wire when the detachment system is traversing a microcatheter.

The slack section can have a first length when the pull wire is in a relaxed condition and a second length when the pull wire is in a tensed condition. The distal end of the pull wire can translate proximally when the slack section exceeds the second length.

The slack section can be a plurality of bends in the pull wire positioned proximal to a location wherein the loop wire contacts the pull wire.

The slack section can be a spiral coil formed into the pull wire proximal to a location wherein the loop wire contacts the pull wire.

The slack section can be a section of stretchable material positioned along a length of the pull wire proximal to a location wherein the loop wire contacts the pull wire.

The pull wire can be defined by a proximal rigid section and a distal rigid section, and the slack section, including the stretchable material, can be positioned between the proximal rigid section and the distal rigid section, wherein the proximal rigid section and the distal rigid section as a lower degree of elasticity than the section of stretchable material.

The section of stretchable material can be a polymeric suture. The detachment system can a key affixed to the implantable medical device proximate a proximal end of the implantable medical device. The key can include a distal opening therethrough, wherein a stretch resistant fiber passes through the distal opening. The key can include a proximal opening therethrough. The key can include a bridge separating the distal opening and the proximal opening. The stretch resistant fiber can be engaged to the key, extended through an implant lumen of the implantable medical device, and affixed to the implantable medical device proximate a distal end of the implantable medical device. The bridge can support a portion of the pull wire in a distal direction from the loop opening. The slack section can be positioned proximal to the key.

A method as described herein can include providing a tubular body comprising a lumen extending therethrough and a compressible distal tube. The method can include affixing a loop wire to the tubular body. The method can include compressing the compressible distal tube. The method can include positioning a loop opening in the loop wire approximate a distal end of the compressible distal tube while the loop wire is affixed to the tubular body such that the loop wire is extended through the lumen. The method can include extending a pull wire through the lumen. The method can include extending the loop opening through a key of an implantable medical device. The method can include extending a distal end of the pull wire through the loop opening. The method can include forming a slack section of the pull wire to inhibit premature detachment of the implantable medical device by inhibiting movement of the distal end of the pull wire when the tubular body is traversing a microcatheter.

The slack section can have a first length when the pull wire is in a relaxed condition and a second length when the pull wire is in a tensed condition, wherein the distal end of the pull wire translates proximally when the slack section exceeds the second length. The method can further include releasing the implantable medical device when the pull wire is retracted such that a final length of the slack section exceeds the second length.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive systems and devices, by way of example only, not by way of limitation.

FIG. 1 is an illustration of a delivery/detachment system and implant, according to aspects of the present invention.

FIGS. 2A and 2B are illustrations of detachment features (i.e., keys) each having a stretch resistant fiber therethrough, according to aspects of the present invention.

FIGS. 3A-3C are illustrations of keys affixed to an embolic coil, according to aspects of the present invention.

FIG. 4 is an illustration of embolic coils being positioned within an aneurysm, according to aspects of the present invention.

FIGS. 5A and 5B are illustrations of example slack sections including a plurality of slack bends, according to aspects of the present invention.

FIGS. 6A and 6B are illustrations of example slack sections including spiral coils, according to aspects of the present invention.

FIGS. 7A and 7B are illustrations of example slack sections including stretchable material, according to aspects of the present invention.

FIGS. 8A-8D illustrate a sequence of steps for releasing an embolic implant from a detachment system, according to aspects of the present invention.

FIG. 9 is a flow diagram illustrating steps for designing, constructing, or configuring a detachment system and implant, according to aspects of the present invention.

### DETAILED DESCRIPTION

An object of the present invention is to decrease the occurrence of or ultimately to prevent premature detachment of an embolic coil from a detachment system prior to placing the coil at a treatment site, i.e., an aneurysm. More specifically, it is an object of the present invention to provide an amount of provide an amount of tolerance to the movement of a pull wire to ensure a distal portion of the pull wire does not inadvertently translate proximally to deploy the embolic implant. Certain current designs for embolic coil delivery systems can include a tubular body having a compressed distal tube that, once released from compression, delivers the embolic coil to a treatment site. Within that distal tube (also referred to herein as a “distal hypotube”) passes both a loop wire and a pull wire. The loop wire can extend into a detachment features (also referred to herein as a “key”) of the implant and loop onto the pull wire to secure the distal hypotube into its compressed state, while also containing the embolic coil that is attached to the key. One common pitfall to prior designs is that there is a chance that the pull wire can prematurely translate proximally from the loop wire, for example as a physician is delivering the device through tortuosity and reactive frictional forces cause the pull wire to retract. Premature detachment of the embolic coil from the detachment system can be a significant problem, as the physician no longer controls the timing/position of the placement of the embolic coil at the aneurysm. The present devices, systems, and methods provide a solution to early, inadvertent deployment of the embolic coil.

Referring to the figures, FIG. 1 is an illustration of a delivery/detachment system 10 and an implantable medical device 12 (which is an embolic coil in the example shown), according to aspects of the present invention. The implantable medical device 12 is also referred to herein as implant 12. The detachment system 10 can include a proximal tube 100, a coiled section 600 comprising a support coil 200, a distal tube 300, a sleeve 500 surrounding the coiled section 600, a loop wire 400 extending through the coiled section 600, and a pull wire 140 extending through the coiled section 600. A distal end 144 of the pull wire 140 can extend at least partially beyond a proximal portion of a key 18 (also referred to herein as a “detachment feature”) of the implant 12. The detachment system 10 can have a tubular body 90 that is formed by the proximal tube 100, the coiled section 600 comprising the support coil 200, and the distal tube 300. When the distal tube 300 is compressed, as will be described below for when the distal hypotube 300 includes a compressible portion 306, the distal tube 300 can be referred to as a compressed distal tube.

A proximal end 102 of the proximal tube 100 can extend proximally within a delivery member (e.g., catheter 250). A distal end 104 of the proximal tube 100 can be connected to a proximal end 202 of the support coil 200. A distal end 204 of the support coil 200 can be connected to the distal tube 300 at one end, and the implant 12 can be connected to the distal tube 300 at the distal end 304 of the distal tube 300. The proximal tube 100 can include a proximal lumen 108, the coiled section 600 and support coil 200 can include a coil lumen 208, and the distal tube 300 can include a distal lumen 308. The proximal lumen 108, coil lumen 208, and distal lumen 308 provide a contiguous lumen through which the pull wire 140 and loop wire 400 pass.

The coiled section 600 can be formed primarily of a non-radiopaque material, such as steel, and can include a radiopaque section 216 made of a radiopaque material, such as platinum and/or tungsten. The radiopaque section 216 can be positioned between a proximal, non-radiopaque section of the support coil 200 and a distal, non-radiopaque section of the support coil 200. The radiopaque section 216 can be positioned a predetermined distance from a distal end 304 of the detachment system 10 so that a physician can readily visualize the placement of the distal portion of the system during a treatment procedure. The proximal section, radiopaque section 216, and distal section of the support coil 200 can be concentrically welded.

The sleeve 500 can cover at least a portion of the flexible section 106 to inhibit deformation of the flexible section and/or reduce friction with vasculature and the flexible section 106 during intravascular navigation. In some examples, the sleeve 500 can cover about 10 cm of the proximal tube 100 approximate and/or including the distal end 104 of the proximal tube 100. When the detachment system 10 is assembled, the coiled section 600 and sleeve 500 can be more flexible than the distal hypotube 300 and the proximal hypotube 100. One way to measure flexibility is to perform a three-point bend test wherein a portion of the detachment system 10 is held fixed at two end points, a force is applied perpendicularly to the detachment system 10 centrally between the points, and flexibility is quantified by the length of deflection of the detachment system 10 caused by the force. When measured this way, in some examples, the coiled section 600 and sleeve 500 can be about 1.5 times more flexible than the distal hypotube 300 and about 20 times more flexible than the proximal hypotube 100. In other words, when the three-point test is performed identically on the three sections 100, 600, 300, the coiled section 600 can deflect over a length that is about 1.5 time the deflection length of the distal hypotube 300 and about 20 times the length of deflection of the proximal hypotube 100. Flexibility can be measured in other ways as would be appreciated and understood by a person of ordinary skill in the art. When the detachment system 10 is assembled, the coiled section 600 and sleeve 500 can be more flexible than the distal hypotube and the proximal hypotube as flexibility is determined by other means as would be known to a person of ordinary skill in the art.

The loop wire 400 can be attached to the detachment system 10 at locations along the tubular body 90. The loop wire 400 can include a first end attachment 406 to connect the loop wire 400 to the wall of the lumen 108, 208, 308 and a second end attachment 408 to connect an opposite end of the loop wire 400 to the wall of the lumen 108, 208, 308. The first end attachment 406 and second end attachment 408 can be welds, adhesives, or other mechanical fasteners that connect the loop wire 400 to the tubular body 90. The first end attachment 406 and second end attachment 408 can be located along the proximal hypotube 100, as shown in FIG. 1, or any other location of the tubular body 90, including along the coiled section 600 or the proximal hypotube 300.

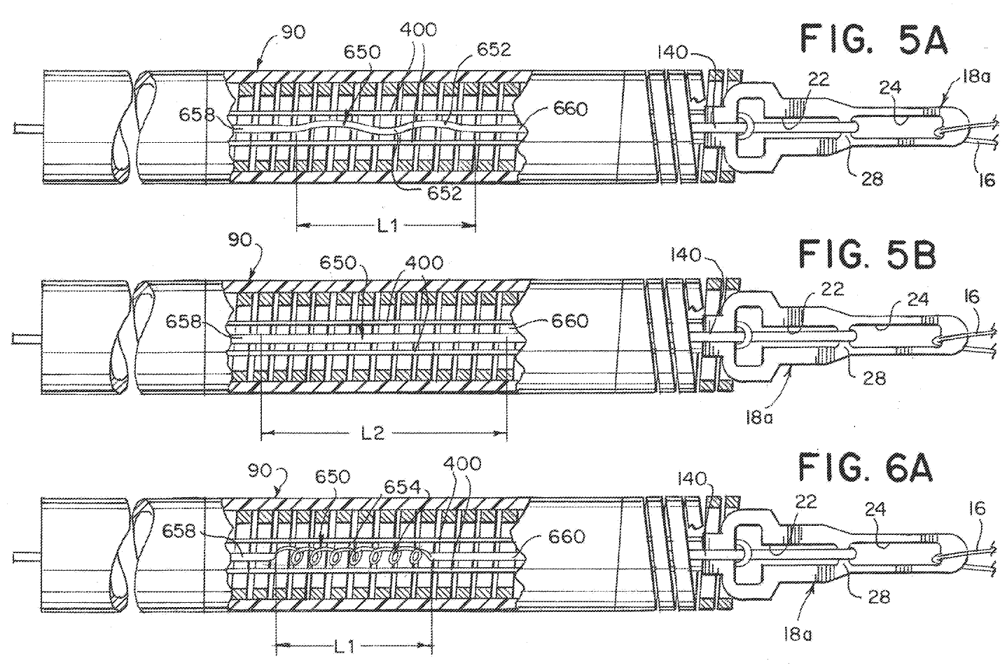

The pull wire 140 can include a slack section 650 positioned along length of the pull wire 140. The slack section 650 can provide an area of the pull wire 140 that can stretch and relax while the detachment system 10 is traversing through a microcatheter (e.g., catheter 250). The slack section 650 can enable the section of the pull wire 140 distal to the slack section (i.e., distal rigid section 660) to remain stationary independent from unintended movement of the pull wire proximal to the slack section (i.e., proximal rigid section 658 or the proximal end 142 of the pull wire 140). The distal rigid section 660 of the pull wire 140 is the section of the pull wire 140 that engages with the loop wire 400 to contain the implant 12. Additional details regarding the loop wire 140 and slack section 650 are provided below with reference to FIGS. 5A to 7B. The example detachment system 10 illustrated in FIG. 1 shows a slack section 650 positioned within the coil lumen 208, which is in accordance with certain examples. The slack section 650 can also be positioned at other locations along the length of the tubular body 90, for example within the proximal lumen 108 or the distal lumen 308.

As described above, one common concern with prior systems is inadvertent proximal translation of the pull wire as the system is deliver through the tortuosity. Inhibiting proximal translations of the distal rigid section 660 pull wire 140 can prevent inadvertent proximal translation of the pull wire 140. The distal tube 300 can be compressed or a portion of the distal tube 300 can be compressed such that, once the pull wire 140 is pulled laterally sufficiently to overcome the slack in the stretchable slack section 650, the distal end 144 of the pull wire 140 is removed from the loop at the end of the loop wire 40, and the compressed portion of the distal tube 300 can expand to deliver the implant 12. FIGS. 7A-7B provide a detailed view of a compressible portion 306 of the distal hypotube 300.

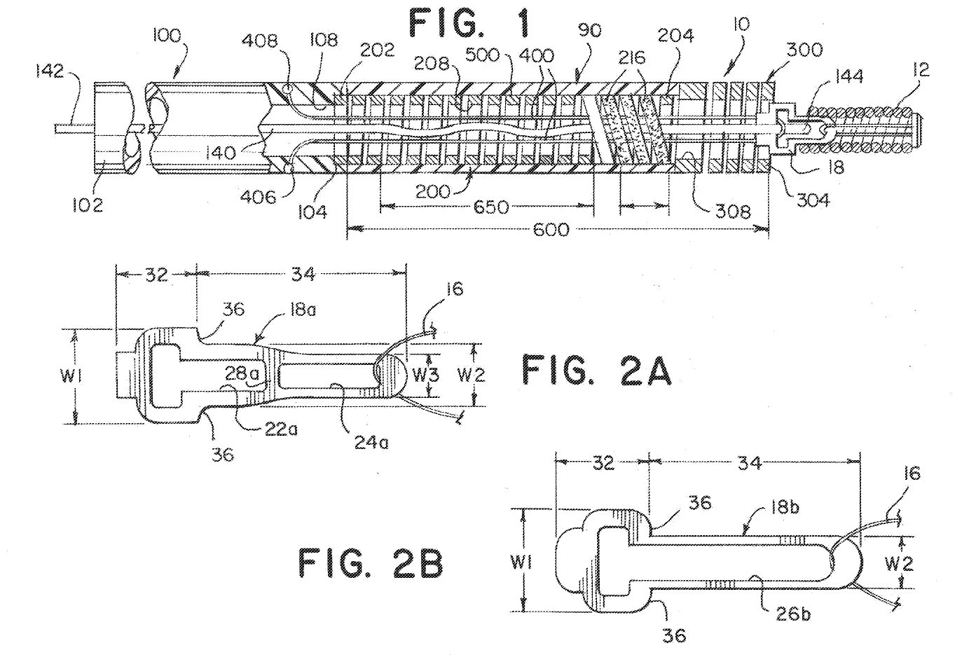

FIGS. 2A and 2B are illustrations of detachment features (i.e., keys 18) each having a stretch resistant fiber 16 therethrough, according to aspects of the present invention. FIG. 2A illustrates a dual opening key 18a having a proximal portion 32 that is sized to engage a mechanical detachment system 10 and/or delivery tube (e.g., the distal hypotube 300). The proximal portion 32 is illustrated as having a width W1. The dual opening key 18a can have a distal portion 34 that is sized to fit within a lumen 13 of the embolic coil (e.g., implant 12). The distal portion 34 can have a wider section having a width W2 that is about as wide as the inner diameter of the implant 12 and a tapered section having a width W3 that is narrower than the inner diameter of the implant 12. The dual opening key 18a can have a proximal tab 38 that is narrower than the proximal portion 32 and is sized to fit within a lumen of a delivery tube (e.g., distal lumen 308). The “dual opening” of the dual opening key 18a can refer to the two separate openings within the face of the key 18a, for example a proximal opening 22 and a distal opening 24. A bridge 28 can separate the proximal opening 22 and the distal opening 24, as illustrated. The bridge 28 can be used to support the distal end 144 of the pull wire 140 when the detachment system 10 is in the loaded/pre-deployed state.

FIG. 2B illustrates a single opening key 18b having a proximal portion 32 that is sized to engage a mechanical detachment system 10 and/or delivery tube (e.g., the distal hypotube 300). The proximal portion 32 is illustrated having a width W1. The single opening key 18b can have a distal portion 34 narrower than the proximal portion 32 and sized to fit within the lumen 13 of the implant 12. The single opening key 18b can have a proximal tab 38 that is narrower than the proximal portion 32 and sized to fit within a lumen of a delivery tube, as also shown for the dual opening key 18a.

When reference is made herein to a key 18, it will be understood to include a dual opening key 18a or a single opening key 18b. After the key 18 is formed, a stretch resistant fiber 16 can be threaded through a distal opening 24 of the dual opening key 18a or the single opening 26 of the single opening key 18b. The stretch resistant fiber, which can be a suture material and the like, can secure the key to the embolic coil portion of the implant. The key 18 can include engagement surfaces 36 at a distal end of the proximal portion 32 of the key 18. This engagement surfaces 36 can abut a proximal end 15 of the implant 12.

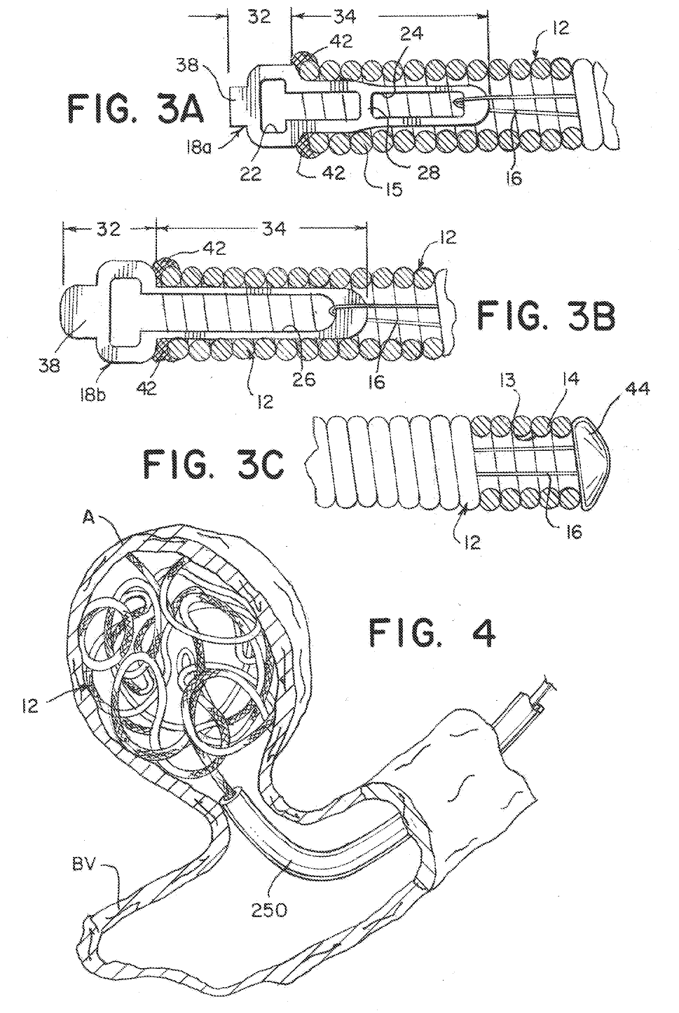

FIGS. 3A-3C are illustrations of keys 18 affixed to an embolic coil (e.g., implant 12), according to aspects of the present invention. In particular, FIGS. 3A and 3B are illustrations of the keys 18 with the distal portion 34 fully inserted into the lumen 13 of the implant 12 and wherein the key 18 is affixed to the implant 12 with welds 42 or other attachments. The welds 52 can be positioned at locations wherein the engagement surfaces 36 of the key 18 meets the proximal end 15 of the implant 12. In both FIGS. 3A and 3B, the key 18 is illustrated having a distal portion 34 that has a width over at least a portion of the length of the distal portion 34 that is about equal to the inner diameter of the lumen 13 of the implant 12.

FIG. 4 is an illustration of embolic coils (e.g., implant 12) being positioned within an aneurysm A, according to aspects of the present invention. The detachment system 10 is passed through a blood vessels BV to the aneurysm A through a catheter 250. Once positioned, the implant(s) 12 can loop and bend within the aneurysm sac to form a thrombotic mass. The implant(s) 12 can loop back on themselves and/or loop next to other implants. As the aneurysm A becomes increasingly packed, overlapping portions of the implant 12 can press into each other.

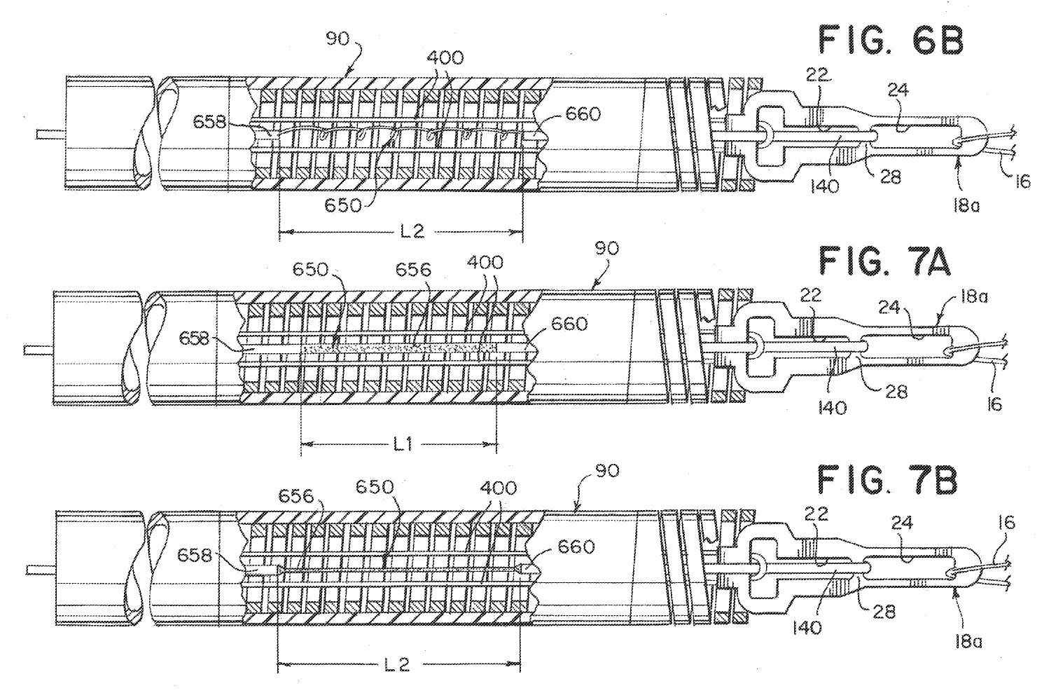

FIGS. 5A to 7B are illustrations of example slack sections 650 in a pull wire 140, according to aspects of the present invention. The pull wire 140 can be a metallic material, such as steel, that has a degree of elasticity that allows the pull wire 140 to not only traverse the tortuosity but to also provide shape memory, and the slack section can be formed into the length of the pull wire 140, as shown in FIGS. 5A and 6A. Referring to FIG. 5A, the slack section 650 can include a series of side-to-side alternating bends 652 that can straighten when stretched and retake their original, bent state when relaxed. FIG. 5A shows the alternating bends 652 in a relaxed condition (i.e., the pull wire 140 has not been pulled proximally), and FIG. 5B shows the alternating bends 652 in a tensed condition (i.e., the pull wire 140 has been pulled proximally). The slack section 650 can have a first length L1 when the pull wire 140 is in a relaxed condition (FIG. 5A) and a second length L2 when the pull wire 140 is in a tensed condition (FIG. 5B). The distal end 144 of the pull wire 140 can translate proximally when the slack section 650 exceeds the second length L2. Stated otherwise, stretching of the slack section 650 between L1 and L2 does not translate the distal rigid section 660, and only extending the slack section 650 beyond L2 will cause the distal and 144 of the pull wire 140 to retract through the opening 405 in the pull wire 400.

Referring to FIG. 6A, the slack section 650 can include a series of spiral coils 654, like a spring, that enables the slack section 650 to stretch and shrink as the proximal rigid portion 658 moves, thereby ensuring the distal rigid section 660 does not prematurely retract from the loop wire 400. FIG. 6A shows the spiral coils 654 in a relaxed condition (i.e., the pull wire 140 has not been pulled proximally), and FIG. 6B shows the spiral coils 654 in a tensed condition (i.e., the pull wire 140 has been pulled proximally). The slack section 650 can have a first length L1 when the pull wire 140 is in a relaxed condition (FIG. 6A) and a second length L2 when the pull wire 140 is in a tensed condition (FIG. 6B). The distal end 144 of the pull wire 140 can translate proximally when the slack section 650 exceeds the second length L2, as stated above.

Referring to FIG. 7A, the slack section 650 can be a stretchable material 656 positioned along a length of the pull wire 140. The stretchable material 656 can include a polymer suture, another synthetic or natural fiber, or the like. Similar to the bends 652 and spiral coils 654 described above, the stretchable material 656 can enable the slack section 650 to stretch and shrink as the proximal rigid portion 658 moves, thereby ensuring the distal rigid section 660 does not prematurely retract from the loop wire 400. The slack section 650 can have a first length L1 when the pull wire 140 is in a relaxed condition (FIG. 7A) and a second length L2 when the pull wire 140 is in a tensed condition (FIG. 7B). The distal end 144 of the pull wire 140 can translate proximally when the slack section 650 exceeds the second length L2, as stated above.

The distal rigid section 660, proximal rigid section 658, and slack section 650 can each comprise the same material along the length of the three sections. For example, the bends 652 and/or spiral coils 654 described above can be manufactured from the same material as the distal rigid section 660 and the proximal rigid section 658. However, the sections can include different materials. For example, the distal rigid section 660 and proximal rigid section 658 can be manufactured from a less-elastic material (i.e., higher Young's modulus) such as steel, chromium alloys, and the like. The slack section can include a more elastic material, such as titanium, nickel alloys, and the like. A more elastic slack section 650 can provide more flection and stretch at portion of the pull wire 140 so as to further decrease the chance of premature proximal translation of the distal rigid section 660 that is engaged with the loop wire 400. In the case of the slack section 650 is a stretchable material 656, the stretchable material 656 can be a different material than the distal rigid section 660 and the proximal rigid section 658.

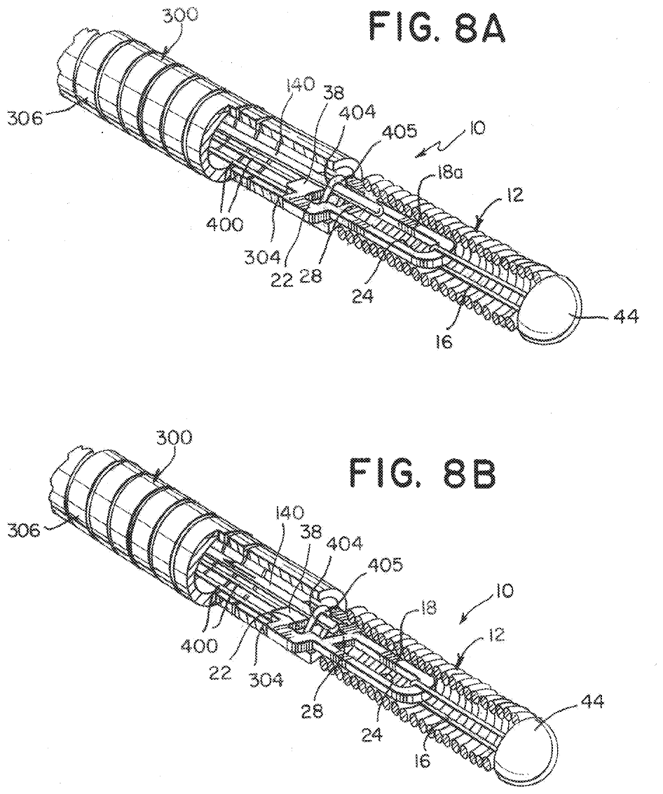

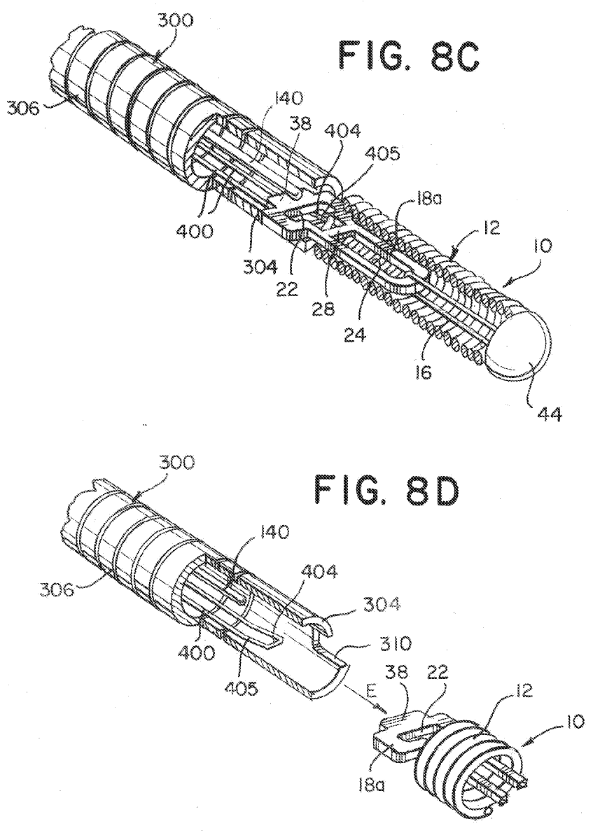

FIGS. 8A-8D illustrate a sequence of steps for releasing an embolic implant 12 from a detachment system 10, according to aspects of the present invention. FIG. 8A is an illustration of the implant 12 and delivery tube (e.g., distal hypotube 300) configured for delivery and positioning of the implant 12. FIGS. 8B through 8D illustrate releasing the example embolic implant 12 from the distal hypotube 300. A portion of the distal hypotube 300 is cut away for illustration purposes. The more proximal features of the tubular body 90 are not shown in the views.

FIG. 8A illustrates the detachment system including a pull wire 140 and a loop wire 400 locked into the key 18 of the implant 12 (the key shown in FIGS. 8A-8D is a dual opening key 18a, but the illustrations could equally apply to a single opening key 18b). The distal tube 300 can include a compressible portion 306. As described above, the slack section 650 can be proximal to the distal tube 300 in the detachment system 10, and the slack section 650 is not visible in the views shown in FIGS. 8A-8D. The loop wire 400 can have an opening 405 at a distal end 404 of the loop wire 400, and the opening 405 can be placed through an opening in the key 18 (e.g., proximal opening 22 in a dual opening key 18a, or the singular opening 26 in a single opening key 18b). When the pull wire 140 is placed through the opening 405, the implant 12 is now secure.

In the case of a dual opening key 18a, the key can include a bridge 28 positioned distally from the loop wire opening 405 and positioned to support a distal portion of the pull wire 140 that is distal of where the loop wire opening 405 wraps around by the pull wire 140. Configured thusly, the bridge 28 can support the distal portion of the pull wire 140 such that when the loop wire 400 tensions against the pull wire 140 at the loop opening 405, the bridge 28 can inhibit the distal portion of the pull wire 140 from deforming. The proximal tab 38 of the key 18 can be positioned to support a portion of the pull wire 140 that is proximal of where the loop wire opening 405 is supported by the pull wire 140. The combination of the bridge 28 and the proximal tab 38 can inhibit the pull wire 140 (i.e., the distal rigid section 660) from deforming due to forces applied by the loop wire 400. The distal hypotube 300 can be detachably attached to the implant 12 as illustrated in FIG. 8A during delivery of the implant 12 through the vasculature and while the implant 12 is being positioned at a treatment site. The bridge 28 can reduce the likelihood that the implant 12 is prematurely released due to bending of the pull wire 140 due to forces from the loop wire 400.

FIG. 8B illustrates the pull wire 140 being drawn proximally to begin the release sequence for the implant 12. As stated above, since the view shows the distal end 144 of the pull wire translating proximally, it means that sufficient tension was placed on the proximal rigid section 658 that the slack section 650 extended beyond L2 (shown in FIGS. 6A and 6B). FIG. 8C illustrates the instant the pull wire 140 exits the opening 405 and is pulled free of the loop wire 400. The distal end 404 of the loop wire 400 falls away and exits the key 18. As can be seen, there is now nothing holding the implant 12 to the distal hypotube 300. FIG. 8D illustrates the end of the release sequence. Here, the compressible portion 306 has expanded/returned to its original shape and “sprung” forward. An elastic force E is imparted by the distal end 304 of the distal hypotube 300 to the implant 12 to “push” it away to ensure a clean separation and delivery of the implant 12. The compressible portion 306 can be a spiral cut portion of the distal hypotube 300, for example a laser cut spiraled segment that can be compressed when the detachment system 10 is loaded.

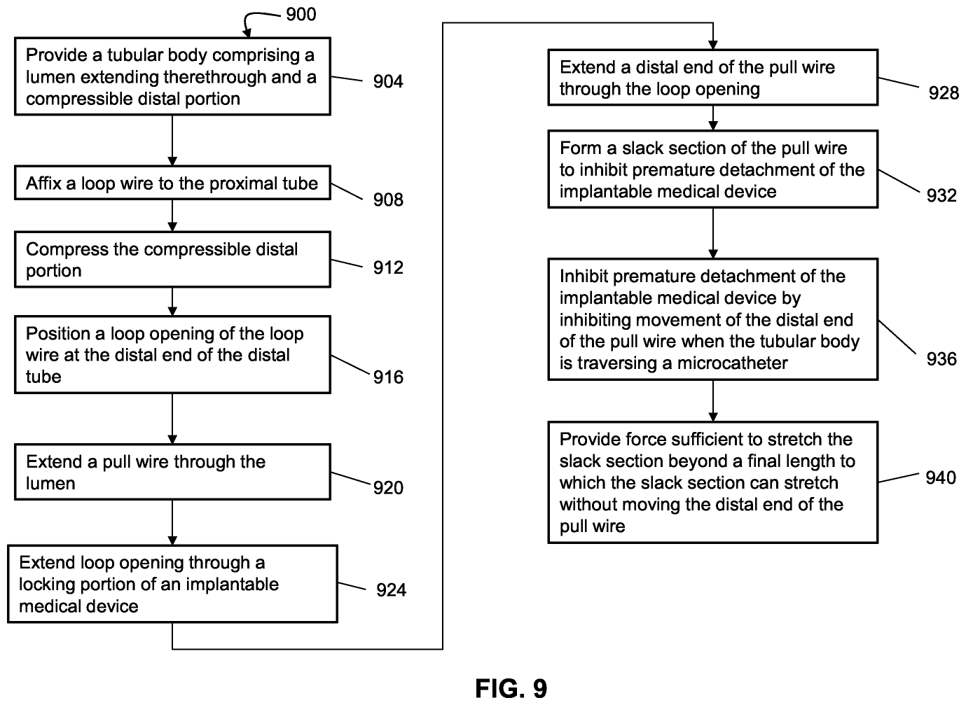

FIG. 9 is a flow diagram illustrating a method 900 for designing, constructing, or configuring a detachment system and implant, according to aspects of the present invention. Steps 904 through 932 describe steps to create/construct one or more of detachment systems 10 described herein. In step 904, the construction of the detachment system 10 can begin with providing a tubular body 90 comprising a lumen (e.g., lumen 108, 208, 308) extending therethrough and a compressible distal tube (e.g., distal hypotube 300). In step 908, a loop wire 400 can be affixed to the tubular body 90. For example, proximal ends of the loop wire can be attached to the tubular body at a first end attachment 406 and a second end attachment 408.

In step 912, the compressible distal tube 300 can be compressed into its loaded configuration. At step 916, a loop wire opening 405 in the loop wire 400 can be positioned proximate a distal end 304 of the compressible distal tube such that the loop wire 400 is extended through the lumen (e.g., lumen 108, 208, 308). In step 920, the pull wire 140 can be extended through the lumen (e.g., lumen 108, 208, 308).

In step 924, the loop opening 405 can be extended through a key 18 of an implantable medical device 12. In step 928, a distal end 144 of the pull wire 140 can be extended through the loop opening 405 of the loop wire 140. At step 932, a slack section 650 can be formed into the pull wire 140. The slack section 650 can be any of the slack sections described herein or any similar slack section that enables the proximal end 142 of the pull wire 140 to translate proximally and distally independently from the distal end 144 of the pull wire 140 when the detachment system is traversing a microcatheter.

The steps for creating/constructing the detachment system 10 can end after step 932. In some examples, steps 936 and 940 provide additional steps to inhibit inadvertent proximal translation of the pull wire 140 and such that the implant can be deployed. For example, in step 936, proximal translation of the pull wire 140 through the loop wire 400 while the implantable medical device 12 is delivered through vasculature to a treatment site can be subdued via inhibiting movement of the distal end 144 of the pull wire 140. In step 940, sufficient force can be provided to stretch the slack section 650 beyond a final length to which the slack section 650 can stretch without moving the distal end 144 of the pull wire 140 (e.g., L2 described above).

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

The descriptions contained herein are examples of embodiments of the invention and are not intended in any way to limit the scope of the invention. As described herein, the invention contemplates many variations and modifications of the implant and methods for making and using the same, including alternative materials, alternative geometries of component parts, alternative positioning of component parts in relation to each other, etc. These modifications would be apparent to those having ordinary skill in the art to which this invention relates and are intended to be within the scope of the claims which follow.

What is claimed is:

1. A detachment system for delivering an implantable medical device to a target location of a body vessel, the system comprising:
a tubular body comprising a lumen extending therethrough and a compressed distal tube;
a loop wire comprising a first end affixed to the tubular body and comprising a loop opening positioned proximate a distal end of the compressed distal tube; and
a pull wire extending through the lumen and through the loop opening, the pull wire being defined by a proximal rigid section, a distal rigid section, and a slack section positioned between the proximal rigid section and the distal rigid section,
wherein the slack section enables the proximal rigid section to translate proximally and distally independently from the distal rigid section when the detachment system is traversing a microcatheter, and
wherein the slack section is a plurality of bends in the pull wire positioned proximal to a location wherein the loop wire contacts the pull wire.

2. The detachment system of claim 1, the slack section has a first length when the pull wire is in a relaxed condition and a second length when the pull wire is in a tensed condition, wherein the distal end of the pull wire translates proximally when the slack section exceeds the second length.

3. The detachment system of claim 1, wherein the slack section is effective to inhibit premature detachment of the implantable medical device by inhibiting movement of the distal end of the pull wire when the detachment system is traversing a microcatheter.

4. The detachment system of claim 1, wherein the plurality of bends is a spiral coil formed into the pull wire.

5. The detachment system of claim 1, wherein the plurality of bends comprises a section of stretchable material positioned along a length of the pull wire proximal to a location wherein the loop wire contacts the pull wire.

6. The detachment system of claim 5, wherein the proximal rigid section and the distal rigid section as a lower degree of elasticity than the section of stretchable material.

7. The detachment system of claim 5, wherein the section of stretchable material is a polymeric suture.

8. The detachment system of claim 1, further comprising:
a key affixed to the implantable medical device proximate a proximal end of the implantable medical device and comprising:
a distal opening therethrough, wherein a stretch resistant fiber passes through the distal opening;
a proximal opening therethrough; and
a bridge separating the distal opening and the proximal opening; and
the stretch resistant fiber engaged to the key, extended through an implant lumen of the implantable medical device, and affixed to the implantable medical device proximate a distal end of the implantable medical device,
wherein the slack section is positioned proximal to the key.

9. The detachment system of claim 1, wherein the plurality of bends are side-to-side alternating bends.

10. A detachment system for delivering an implantable medical device to a target location of a body vessel, the system comprising:
a pull wire extending through a tubular body of the detachment system, the pull wire being defined by a proximal rigid section, a distal rigid section, and a slack section positioned between the proximal rigid section and the distal rigid section; and
a loop wire looped over the pull wire at a distal end of the loop wire,
wherein the slack section is positioned proximal to a loop opening in the loop wire, the slack section being effective to inhibit premature detachment of the implantable medical device by inhibiting movement of a distal end of the pull wire when the detachment system is traversing a microcatheter, and wherein the slack section is a plurality of bends in the pull wire positioned proximal to a location wherein the loop wire contacts the pull wire.

11. The detachment system of claim 10, wherein the slack section has a first length when the pull wire is in a relaxed condition and a second length when the pull wire is in a tensed condition, wherein the distal end of the pull wire translates proximally when the slack section exceeds the second length.

12. The detachment system of claim 10, wherein the plurality of bends are side-to-side alternating bends.

13. The detachment system of claim 10, wherein the plurality of bends is a spiral coil formed into the pull wire proximal to a location wherein the loop wire contacts the pull wire.

14. The detachment system of claim 10, wherein the plurality of bends comprises a section of stretchable material positioned along a length of the pull wire proximal to a location wherein the loop wire contacts the pull wire.

15. The detachment system of claim 14, wherein the proximal rigid section and the distal rigid section comprise a lower degree of elasticity than the section of stretchable material.

16. The detachment system of claim 14, wherein the section of stretchable material is a polymeric suture.

17. The detachment system of claim 10, further comprising:
a key affixed to the implantable medical device proximate a proximal end of the implantable medical device and comprising:
a distal opening therethrough, wherein a stretch resistant fiber passes through the distal opening;
a proximal opening therethrough; and
a bridge separating the distal opening and the proximal opening; and
the stretch resistant fiber engaged to the key, extended through an implant lumen of the implantable medical device, and affixed to the implantable medical device proximate a distal end of the implantable medical device,
wherein the bridge supports a portion of the pull wire in a distal direction from the loop opening, and
wherein the slack section is positioned proximal to the key.

18. A method comprising:
providing a tubular body comprising a lumen extending therethrough and a compressible distal tube;
affixing a loop wire to the tubular body;
compressing the compressible distal tube;
positioning a loop opening in the loop wire approximate a distal end of the compressible distal tube while the loop wire is affixed to the tubular body such that the loop wire is extended through the lumen;
extending a pull wire through the lumen;
extending the loop opening through a key of an implantable medical device;
extending a distal end of the pull wire through the loop opening; and
forming a slack section of the pull wire to inhibit premature detachment of the implantable medical device by inhibiting movement of the distal end of the pull wire when the tubular body is traversing a microcatheter, wherein the slack section is a plurality of bends in the pull wire positioned proximal to a location wherein the loop wire contacts the pull wire.

19. The method of claim 18, wherein the slack section has a first length when the pull wire is in a relaxed condition and a second length when the pull wire is in a tensed condition, wherein the distal end of the pull wire translates proximally when the slack section exceeds the second length, the method further comprising:
releasing the implantable medical device when the pull wire is retracted such that a final length of the slack section exceeds the second length.
